(19) United States
(12) Patent Application Publication
(10) Pub. No.: US 2025/0137058 A1
(43) Pub. Date: May 1, 2025

(54) IMMUNOTHERAPY TARGETING

(71) Applicant: The Regents of the University of California, Oakland, CA (US)

(72) Inventors: Hannah Kathryn Carter, San Diego, CA (US); Meghana Sai Pagadala, La Jolla, CA (US); Jorge Silvio Gutkind, La Jolla, CA (US); Victoria Huannyun Wu, La Jolla, CA (US); Maurizio Zanetti, La Jolla, CA (US)

(21) Appl. No.: 18/706,182

(22) PCT Filed: Nov. 1, 2022
(86) PCT No.: PCT/US2022/048508

§ 371 Date: Apr. 30, 2024

Related U.S. Application Data
(60) Provisional application No. 63/274,672, filed on Nov. 2, 2021.

(51) Int. Cl. C12Q 1/6886; A61K 45/06

(52) U.S. Cl. CPC C12Q 1/6886; A61K 45/06; C12Q 2600/156

(57) ABSTRACT

Provided herein are methods of improving sensitivity to immune checkpoint blockade (ICB) in a subject with a solid tumor cancer, the method comprising: administering to the subject with a solid tumor cancer an inhibitor of a tumor immune microenvironment (TIME) gene, wherein the TIME gene is selected from the group consisting of SLC11A1, CCBL2, LILRB2, LILRB4, LAIR1, CTSS, CTSW, B2M, HAUS1, MS4A4A, FCGR2B, LYZ, and GNPTAB, thereby improving sensitivity to ICB in the subject with a solid tumor cancer.

### CROSS-REFERENCE TO RELATED APPLICATIONS

This application claims priority to U.S. Provisional Patent Application No. 63/274,672, filed on Nov. 2, 2021. The disclosure of the prior application is considered part of the disclosure of this application, and is incorporated herein by reference in its entirety.

### FEDERALLY SPONSORED RESEARCH OR DEVELOPMENT

This invention was made with government support under Grant Nos. CA220009, CA269919 and CA247168 awarded by the National Institutes of Health. The government has certain rights in the invention.

### BACKGROUND

Cancer is a disease characterized by heterogeneous somatic and germline mutations that promote abnormal cellular growth, evasion from the immune system, dysregulation of cellular energetics, and inflammation. Immunotherapy has emerged as a promising treatment; however, response rates are low and the determinants of response remain elusive. The potential of galvanizing the immune system is still unmet due to an incomplete understanding of the complex tumor immune microenvironment (TIME). In particular, knowledge of germline factors and other intrinsic factors that interact with characteristics of tumors to render them sensitive to host-immunity or immunotherapy is lacking.

### SUMMARY

The present disclosure is based, at least in part, on methods to discover immunotherapy targets, using specific single nucleotide polymorphisms (SNPs).

Provided herein are methods of improving sensitivity to immune checkpoint blockade (ICB) in a subject with a solid tumor cancer, the method comprising: administering to the subject with a solid tumor cancer an inhibitor of a tumor immune microenvironment (TIME) gene, wherein the TIME gene is selected from the group consisting of SLC11A1, CCBL2, LILRB2, LILRB4, LAIR1, CTSS, CTSW, B2M, HAUS1, MS4A4A, FCGR2B, LYZ, and GNPTAB, thereby improving sensitivity to ICB in the subject with a solid tumor cancer.

In some embodiments, the TIME gene comprises a single nucleotide polymorphism (SNP). In some embodiments, the TIME gene comprises CTSS.

In some embodiments, the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma or prostate cancer. In some embodiments, the solid tumor cancer is follicular lymphoma.

Also provided herein are methods of treating a solid tumor cancer in a subject by detecting a germline tumor immune microenvironment-single nucleotide polymorphism (TIME-SNP) of a TIME gene in a subject, the method comprising: (a) administering to the subject an inhibitor of the TIME gene selected from the group consisting of SLC11A1, CCBL2, LILRB2, LILRB4, LAIR1, CTSS, CTSW, B2M, HAUS1, MS4A4A, FCGR2B, LYZ, and GNPTAB; and (b) administering to the subject an immune checkpoint blockade (ICB) treatment, thereby treating the tumor in the subject.

In some embodiments, the TIME gene comprises CTSS. In some embodiments, the TIME gene is relevant to the solid tumor cancer. In some embodiments, the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma or prostate cancer. In some embodiments, the solid tumor cancer is follicular lymphoma.

In some embodiments, the inhibitor of the TIME gene and the ICB treatment are administered simultaneously. In some embodiments, the inhibitor of the TIME gene is administered prior to the ICB treatment.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1C show identifying heritable characteristics of the tumor immune microenvironment. (FIG. 1A) Overview of the TIME germline analysis. (FIG. 1B) Clustermap depicting 733 IP components and their pairwise correlation across 30 tumor types in the TCGA. (FIG. 1C) Horizontal barplot of variance in phenotype explained by variance in genotype (Vg/Vp) for 235 immune genes estimated separately genome-wide excluding the HLA locus (left panel) and using only the HLA locus (right panel).

FIGS. 2A-2C show detecting putative germline modifiers of the tumor immune microenvironment. (FIG. 2A) Locuszoom plot summarizing 890 associations between TIME eQTLs and 93 immune genes. Outer ring represents locations of all 157 tested IP components. Links show implications in cancer risk, survival or immunotherapy response. (FIG. 2B) Significant associations between TIME eQTLs and 17 genes in the HLA region detected through conditional GWAS analysis for effects on gene expression using either a basic alignment to the reference genome (conditional), or allele specific expression obtained by aligning to a patient-specific HLA reference allele set. (FIG. 2C) Ideogram plot of TIME eQTLs implicated by the discovery analysis and literature curation.

FIGS. 3A-3H show TIME eQTLs underlying antigen presentation stratify melanoma and prostate cancer risk. (FIG. 3A) Violinplot of melanoma PRS trained on UK Biobank and validated in melanoma cases and controls in High Density Melanoma cohort. (FIG. 3B) Odds of melanoma risk among individuals in the top and bottom 10th quantile of PRS in High Density Melanoma cohort. (FIG. 3C) Top 15 TIME eQTLs of feature importance in melanoma PRS. (FIG. 3D) Violinplot of prostate cancer PRS trained on UK Biobank and validated in prostate cancer cases and controls in ELLIPSE Consortium. (FIG. 3E) Odds of prostate cancer risk among individuals in the top and bottom 10th quantile of PRS in ELLIPSE consortium (FIG. 3F) Top 15 TIME eQTLs of feature importance in prostate cancer PRS. (FIG. 3G) Boxplot of M1 and M2 macrophage infiltration in primary TCGA SKCM (melanoma) in top and bottom 10th quantile of melanoma PRS. (FIG. 3H) Boxplot of CD8+ T cell and CD4+ T regulatory cell infiltration in TCGA SKCM (melanoma) in top and bottom 10th quantile of melanoma PRS.

FIGS. 4A-4F show TIME eQTLs associated with survival implicate immune evasion (FIG. 4A) Cox Proportional-Hazards odds ratios for cancer type-specific polygenic survival score (PSS) with overall survival separated by TCGA cancer type. (FIG. 4B) Cox Proportional-Hazards odds ratios for cancer type-specific PSS with progression free survival separated by TCGA cancer type. (FIG. 4C) Overall survival Kaplan-Meier curve based on LUAD PSS in TCGA LUAD. (FIG. 4D) Overall survival Kaplan-Meier curve based on LUAD PSS in SHERLOCK. (FIG. 4E) Cox Proportional Hazards for LUAD PSS in TCGA LUAD and SHERLOCK. (FIG. 4F) Top 15 TIME eQTLs of feature importance in LUAD PSS. High indicates top 25%. Med indicates middle 50% and Low indicates lowest 25% of PSS. Error bars represent standard error of Cox Proportional Hazards model.

FIGS. 5A-5H show TIME eQTLs implicate targets for modulating immune responses. (FIG. 5A) Boxplot of polygenic ICB score (PICS) constructed in melanoma ICB cohort validated in Miao et al. cohort. (FIG. 5B) Boxplot of PICS constructed in melanoma ICB cohort validated in Rizvi et al. cohort. (FIG. 5C) ROC-AUC Curve Analysis for PICS trained on the discovery melanoma cohort with XGBoost based model and tested on Miao et al and Rizvi et al. (FIG. 5D) Top 15 TIME eQTLs of feature importance in PICS. (FIG. 5E) Grid plot of log odds ratio of variants with responder status in 6 ICB cohorts with beta coefficients of classic ICB biomarkers (TMB, PD-L1, PD-1, CTLA-4) association with responder status. (FIG. 5F) Tumor growth curve for C57BL/6 mice implanted with MC38 treated with anti-PD-1. anti-CTSS, and combination of anti-PD-1 and anti-CTSS. (FIG. 5G) Survival curve for C57BL/6 mice implanted with MC38 treated with anti-PD-1, anti-CTSS, and combination of anti-PD-1 and anti-CTSS. (FIG. 5H) Barplot of the proportion of F4/80 Macrophages that are Arginase⁺ M2 macrophages and MHCII⁺ M1 macrophages respectively for MC38 tumors treated with anti-CTSS compared to control.

FIGS. 6A-6C show characterization of TIME eQTLs using in genetic models (FIG. 6A) Cancer relevant associations by category with barplot showing the total number of genes implicated by polygenic risk scores (PRSs), polygenic survival score (PSS) and polygenic ICB score (PICS). (FIG. 6B) Mean enrichment ratio of genetic model immune microenvironment variants in histone marks with corresponding enrichment ratios in specific cell types. (FIG. 6C) Barplot of cell-type specific TIME eQTLs implicated by DICE and ieQTL analysis.

FIG. 7 shows characterization of genes implicated by PICS model TIME eQTLs. A map of TIME eQTL biological functions, immune functions and cancer associations for 15 genes implicated as modifiers of immune checkpoint blockade response. Innate immune function indicates that TIME eQTLs are also DICE eQTLs for macrophages, monocytes or dendritic cells. Adaptive immune function indicates that TIME eQTLs are also DICE eQTLs for CD8+ T cells, CD4+ T cells or B cells. Risk indicates whether a gene was also implicated in PRS models. Survival indicates whether a gene was also implicated in PSS models. Asterisks (*) indicates that a small molecule inhibitor has been reported for a gene.

### DETAILED DESCRIPTION

This disclosure describes methods to discover immunotherapy targets, using specific single nucleotide polymorphisms (SNPs).

Various non-limiting aspects of these methods are described herein, and can be used in any combination without limitation. Additional aspects of various components of methods for modulating gene expression are known in the art.

It must be noted that, as used in the specification and the appended claims, the singular forms “a,” “an” and “the” include plural referents unless the context clearly dictates otherwise.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “biological sample” refers to a sample obtained from a subject for analysis using any of a variety of techniques including, but not limited to, biopsy, surgery, and laser capture microscopy (LCM), and generally includes cells and/or other biological material from the subject. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

Biological samples can also include immune cells. Sequence analysis of the immune repertoire of such cells, including genomic, proteomic, and cell surface features, can provide a wealth of information to facilitate an understanding the status and function of the immune system. Examples of immune cells in a biological sample include, but are not limited to, B cells (e.g., plasma cells), T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, a lymph node sample, living cells, cultured cells, a clinical sample such as, for example, whole blood or blood-derived products, blood cells, or cultured tissues or cells, including cell suspensions.

As used herein, the terms “cancer” and “tumor”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, hematopoietic cancers can include leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, precancerous pathology such as myelodysplastic syndromes, acquired aplastic anemia, Fanconi anemia, paroxysmal nocturnal hemoglobinuria (PNH) and 5q-syndrome and the like.

As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### A. Immunotherapy

Germline variation is responsible for a considerable proportion of variation in immune traits in healthy populations. In the context of tumors, germline variants have the potential to influence immune infiltration, antigen presentation and immunotherapy responses. Autoimmune germline variants modify immunotherapy (e.g., immune checkpoint blockade (ICB)) response and variants underlying leukocyte genes predict tumor recurrence in cancer patients (e.g., breast cancer). For example, the common single nucleotide polymorphism (SNP) rs351855 in FGFR4 was found to suppress cytotoxic CD8+ T cell infiltration and promote higher immunosuppressive regulatory T cell levels via increased STAT3 signaling in murine models of breast and lung cancer.

Efforts to identify germline variation influencing anti-tumor immune responses have pointed to effects on immune infiltration levels and immune pathways, such as TGF-β and IFN-γ. Expression quantitative trait locus (eQTL) analysis is an approach to identify regulatory mechanisms of variants identified by genome-wide association studies (GWASs) and to identify genetic variants that affect the expression of one or more genes. These studies provide evidence that variants may act through specific effects on immune cells. For example, eQTL profiling of 15 sorted immune cell subsets from healthy individuals found that the effects of many eQTLs were specific to immune cell subsets. Understanding mechanisms and cell-type effects of tumor immune microenvironment (TIME) host genetic interactions could identify needed prognostic biomarkers for ICB response, implicating targetable cell types and molecules to boost ICB response rates.

As used herein, “immunotherapy” refers to a treatment of disease (e.g., cancer) by activating or suppressing the immune system. For example, cancer immunotherapy uses the immune system and its components to mount an anti-tumor response through immune activation. In some embodiments, an immunotherapy can include an immune checkpoint inhibitor, an oncolytic virus therapy, a cell-based therapy, a CAR-T cell therapy, or a cancer vaccine. In some embodiments, an immunotherapy can include immune checkpoint blockade (ICB), wherein an immune checkpoint inhibitor is administered. Immune checkpoint blockade (ICB) is a treatment that uses immune checkpoint inhibitors to address a disease (e.g., cancer), wherein the immune checkpoint inhibitor blocks a checkpoint protein from binding to its partner protein or receptor.

In some embodiments, the immunotherapy includes administration of an immune checkpoint inhibitor. In some embodiments, the immune checkpoint inhibitor is a PD-1 inhibitor. Examples of a PD-1 inhibitor can include, but are not limited to, pembrolizumab, nivolumab, cemiplimab, JTX-4014, spartalizumab, camrelizumab, sintilimab, tislelizumab, toripalimab, and dostarlimab. In some embodiments, the immune checkpoint inhibitor is a PD-L1 inhibitor. Examples of a PD-L1 inhibitor can include, but are not limited to, atezolizumab, avelumab, durvalumab, KN035, CK-301, AUNP12, CA-170, and BMS-986189. In some embodiments, the immune checkpoint inhibitor is a CTLA-4 inhibitor (e.g., ipilimumab, tremelimumab). In some embodiments, the immune checkpoint inhibitor is a CTLA-4 inhibitor used in combination with a PD-1 inhibitor or a PD-L1 inhibitor. In some embodiments, the immune checkpoint inhibitor can be any checkpoint inhibitor, e.g., as described in Mazzarella et al., Eur J Cancer (2019) 117:14-31, hereby incorporated by reference.

Provided herein are methods of identifying an immunotherapy target that include (a) selecting a plurality of tumor immune microenvironment (TIME) genes; (b) identifying a cancer relevant TIME gene from the plurality of TIME genes; and (c) determining response of the cancer relevant TIME gene to immunotherapy. In some embodiments, the method can further include detecting the cancer relevant TIME gene in a biological sample from the subject, thereby predicting the subject's response to immunotherapy. In some embodiments, the method can further include obtaining a biological sample from the subject and detecting the cancer relevant TIME gene in the biological sample. In some embodiments, the immunotherapy comprises immune checkpoint blockade treatment.

Also provided herein are methods of improving sensitivity to immune checkpoint blockade (ICB) in a subject that include (a) select a plurality of tumor immune microenvironment (TIME) genes; (b) identify a cancer relevant TIME gene from the plurality of TIME genes; and (c) administering to the subject an inhibitor of the cancer relevant TIME gene, thereby improving sensitivity to ICB in the subject. As used herein, the term “improve sensitivity” can refer to increasing response rates to a specific treatment (e.g., immunotherapy). In some embodiments, improving sensitivity to ICB treatment can refer to inhibiting negative regulatory immune checkpoints or stimulating activating immune checkpoints. In some embodiments, the method can further include administering an immunotherapy to the subject. In some embodiments, the inhibitor of the cancer relevant TIME gene and the immunotherapy can be administered simultaneously. In some embodiments, the inhibitor of the cancer relevant TIME gene can be administered before administering the immunotherapy.

### B. Tumor Immune Microenvironment (TIME)

As used herein, a “tumor immune microenvironment (TIME)” refers to the microenvironment formed by immune cells and their products in tumor tissues. The TIME plays a decisive role in the response of immune checkpoint inhibitors, wherein some immune cells activate anti-cancer immune responses. For example, a patient with higher tumor-infiltrating CD8+ T cells may have an improved response to an immune checkpoint blockade treatment. Therefore, understanding mechanisms and cell-type effects of TIME host genetic interactions could identify needed prognostic biomarkers for ICB response, implicating targetable cell types and molecules to boost ICB response rates.

In some embodiments, genotype analysis to identify germline determinants underlying the tumor immune microenvironment can be used to identify putative new targets for immunotherapy. In some embodiments, genotype analysis can be used to identify a cancer relevant TIME gene that includes a tumor immune microenvironment-single nucleotide polymorphisms (TIME-SNP), wherein the TIME-SNP can be predictive of immunotherapy response in a cancer (e.g., melanoma, non-small cell lung cancer and renal cell carcinoma). In some embodiments, the cancer relevant TIME gene comprising the TIME-SNP can be linked to gene expression in macrophages, including several genes involved in macrophage polarization.

In some embodiments, a cancer relevant TIME gene comprises an immunomodulator, an antigen presentation gene, an immune checkpoint gene, an immune cell-type marker gene, a TGF-β pathway gene, an INF-γ pathway gene, or an immune cell infiltration gene. In some embodiments, the cancer relevant TIME gene is located in the HLA region. In some embodiments, the cancer relevant TIME gene is located outside the HLA region. In some embodiments, the cancer relevant TIME gene comprises a germline variant. In some embodiments, the germline variant comprises a single nucleotide polymorphism (SNP). In some embodiments, the cancer relevant TIME gene comprises SLC11A1, CCBL2, LILRB2, LILRB4, LAIR1, CTSS, CTSW, B2M, HAUS1, MS4A4A, FCGR2B, LYZ, or GNPTAB. In some embodiments, the cancer relevant TIME gene comprises CTSS, TREX1, DHFR, ERAP1, ERAP2, GPLD1, DYNLT1, PSMD11, CTSW, FAM216A, LYZ, C3ARI, DCTN5, DBNDD1, or FPR1. In some embodiments, the cancer relevant TIME gene comprises CTSS. In some embodiments, the cancer relevant TIME gene is relevant to a solid tumor cancer. In some embodiments, the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, the solid tumor cancer is follicular lymphoma.

### C. Method of Treatment

Provided herein are methods of improving sensitivity to immune checkpoint blockade (ICB) in a subject with a solid tumor cancer, the method including: administering to the subject with a solid tumor cancer an inhibitor of a tumor immune microenvironment (TIME) gene, wherein the TIME gene is selected from the group consisting of SLC11A1, CCBL2, LILRB2, LILRB4, LAIR1, CTSS, CTSW, B2M, HAUS1, MS4A4A, FCGR2B, LYZ, and GNPTAB, thereby improving sensitivity to ICB in the subject with a solid tumor cancer.

In some embodiments, the method can further include obtaining a biological sample of the subject. In some embodiments, the method can further include detecting the TIME gene in the biological sample, wherein the detection of the TIME gene can be used to predict the subject's response to a treatment (e.g., immunotherapy).

In some embodiments, the TIME gene comprises a germline TIME-SNP, wherein the germline TIME-SNP is a germline variant of the TIME gene comprising a single nucleotide polymorphism (SNP). In some embodiments, the TIME gene comprises a single nucleotide polymorphism (SNP).

In some embodiments, the TIME gene comprises CTSS. In some embodiments, the TIME gene is relevant to a solid tumor cancer. In some embodiments, the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma or prostate cancer. In some embodiments, the solid tumor cancer is follicular lymphoma.

Also provided herein are methods of treating a solid tumor cancer in a subject by detecting a germline tumor immune microenvironment-single nucleotide polymorphism (TIME-SNP) of a TIME gene in a subject, the method including: (a) administering to the subject an inhibitor of the TIME gene selected from the group consisting of SLC11A1, CCBL2, LILRB2, LILRB4, LAIR1, CTSS, CTSW, B2M, HAUS1, MS4A4A, FCGR2B, LYZ, and GNPTAB; and (b) administering to the subject an immune checkpoint blockade (ICB) treatment, thereby treating the tumor in the subject.

In some embodiments, the inhibitor of the TIME gene and the ICB treatment are administered simultaneously. In some embodiments, the inhibitor of the TIME gene is administered prior to the ICB treatment.

### EXAMPLES

The disclosure is further described in the following examples, which do not limit the scope of the disclosure described in the claims.

### Example 1—Identifying Heritable Characteristics of the Tumor Immune Microenvironment

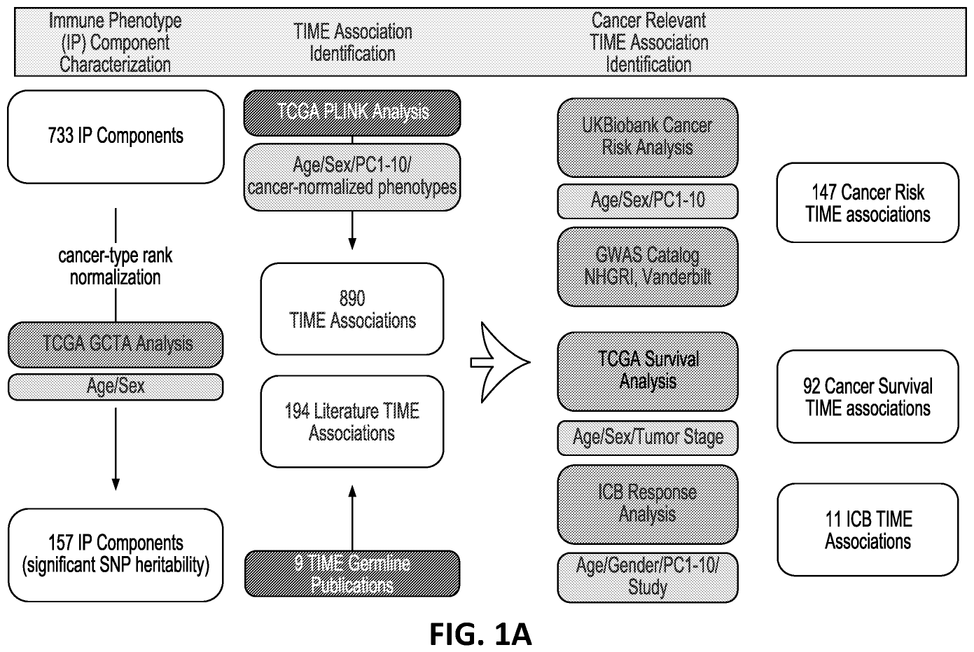

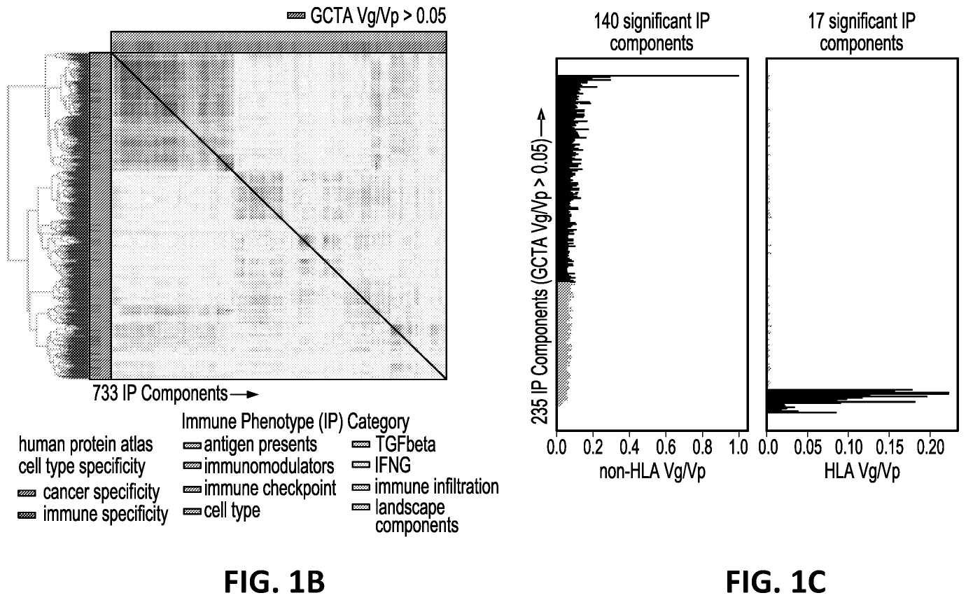

Germline determinants underlying the tumor immune microenvironment were used to identify putative new targets for immunotherapy. Genotype data from the Cancer Genome Atlas (TCGA) was used to identify 532 TIME-SNPs and was further analyzed to converge on a subset that influenced cancer outcomes. These TIME-SNPs were predictive of immunotherapy response in melanoma, non-small cell lung cancer and renal cell carcinoma human cohorts and many were linked to gene expression in macrophages, including several genes involved in macrophage polarization (FIG. 1A).

To focus on common germline genetics with the potential to modify tumor immune responses, it was assessed to see which characteristics of the TIME showed evidence of SNP heritability. To describe the TIME, a comprehensive set of immune phenotype (“IP”) components were collected comprising composite measures derived from bulk gene expression and expression levels of individual immune-related genes (FIG. 1B). Composite phenotypes included infiltrating immune cell levels calculated using CIBERSORTx (immune infiltrates) and 6 immune subtype scores from a pan-cancer TCGA analysis by Thorsson et al. (landscape components). Immunomodulators were collected from Thorsson et al., where weighted gene correlation network analysis was used as an unbiased systematic approach to identify gene sets relevant to the TIME. Genes from these sets were included along with immune checkpoint genes, cell type markers, antigen presentation genes, TGF-β pathway genes, and IFN-γ genes as these have been implicated as important modifiers of the TIME. After removing IP components with high numbers of zero values to reduce spurious associations, 724 immune-related genes and 9 composite phenotypes (733 IP components total) measured across 30 cancer types were retained. Each IP component (gene expression level or composite phenotype) was analyzed independently.

The potential of germline variation was evaluated to explain inter-tumor differences in IP components by performing SNP heritability analysis (FIG. 1A). Since highly polymorphic regions such as the HLA locus can inflate SNP heritability estimates, SNP heritability attributable to the HLA locus and the rest of the genome was separately estimated. 235 (32.0%) IP components were identified where levels were SNP-heritable (FIG. 1C). No composite phenotypes passed heritability thresholds and thus remaining associations were with gene expression and will be referred to as TIME eQTLs. For these 235 genes, 2-state GCTA analysis was conducted and identified 140 (59.6%) that had a significant proportion of SNP heritability attributable to regions outside the HLA locus, while 17 (7.2%) were mostly attributable to the HLA locus. The TIME eQTL discovery analysis was focused on these 157 heritable immune genes.

### Example 2—Detecting Putative Germline Modifiers of the Tumor Immune Microenvironment

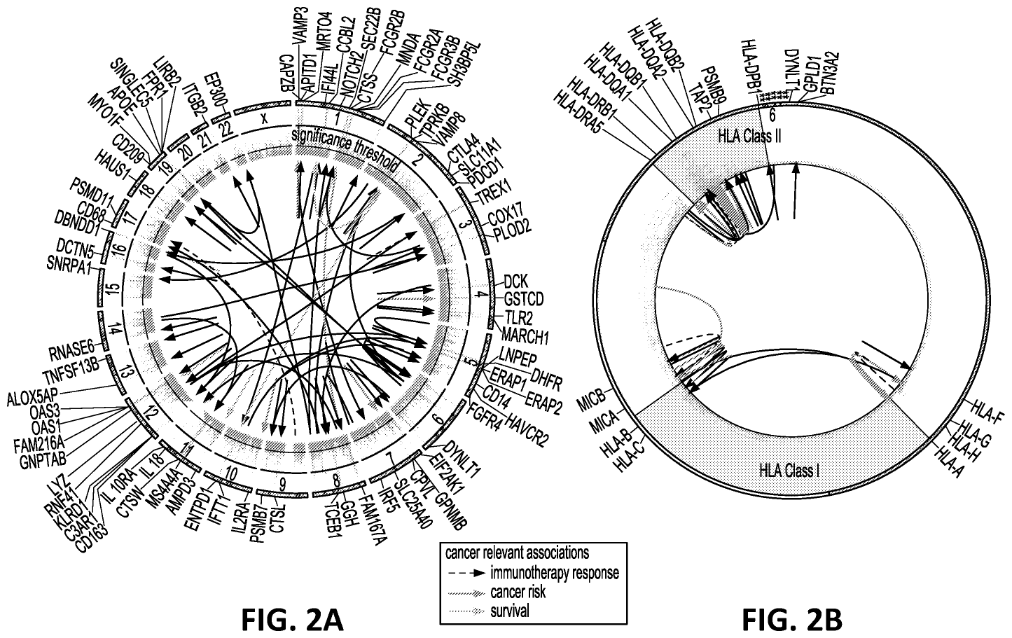

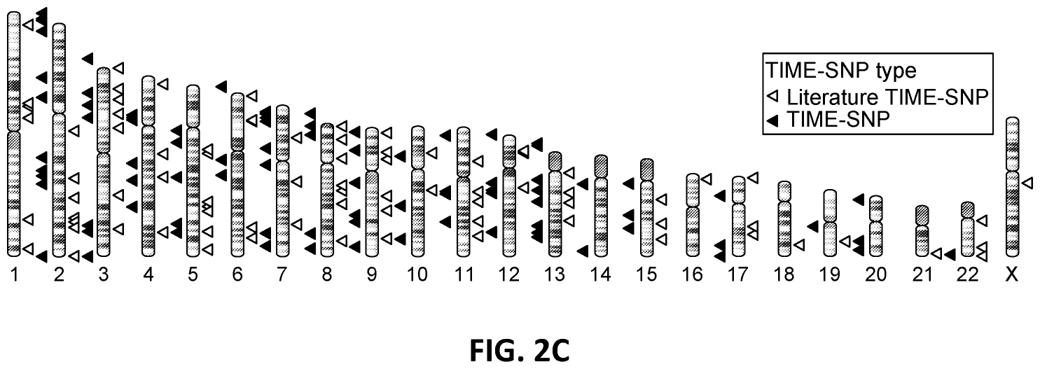

To identify TIME eQTLs, a genome-wide association study (GWAS) was performed. First, each of the 140 heritable immune genes outside of the HLA locus across individuals of European ancestry in the TCGA were analyzed. Immune gene expression was inverse-rank normalized within tumor type, such that tumor-type specific differences were removed. Only common germline variants with minor allele frequency>1% were considered and imputation quality (Rsq) was evaluated to ensure high accuracy. No evidence of inflation was observed. Using linkage and distance-based clumping, 825 TIME eQTLs were identified (FIG. 2A). Cis associations, defined as an associated locus occurring within 1 MB of a gene transcription start site, encompassed the majority (95.0%) of associations. while 5.0% of the associations were trans. Mechanisms of trans associations are complex and tend to have weaker effects on transcriptional regulation. In contrast, cis associations are proximal to an IP component and have more direct effects on transcription. Overall, ERAP2 (181, 21.9%), CCBL2 (76, 9.2%), DHFR (75, 9.0%) and ERAP1 (70, 8.5%) had the most germline associations of the 140 genes tested.

To remove HLA region associations solely attributable to LD structure, conditional GWAS analysis was conducted for seventeen genes in the HLA region of chromosome 6. Alignment to a general HLA gene reference can introduce error into expression level estimates due to the highly polymorphic nature of these genes. Therefore, SNP associations with gene expression estimates derived from allele-specific RNA alignments were revisited and GWAS analysis was performed using allele specific expression. In total, 65 TIME eQTLs in the HLA region were identified (FIG. 2B). Generally, LD-independent eQTLs clustered by genomic regions with HLA-A, HLA-B, HLA-C associated variants falling in the MHC Class I genomic region and HLA-DQB1, HLA-DQA1, HLA-DPB1, HLA-DRB5 associated variants falling in the MHC Class II genomic region. Combining GWAS and conditional HLA GWAS associations, 890 TIME eQTLs were identified.

It was noted that there was some correlation among immune genes across tumors, especially those related to macrophages and lymphocytes which were the most abundant infiltrating immune cells. The largest group of correlated genes included MHC Class I and II genes along with macrophage genes VSIG4, CD163, FCGR2A FCGR3A, HAVCR2, LILRB2, LILRB4 and CD53 and was most strongly associated with antigen presentation, dendritic cell processing, and IL-10 production. The next largest comprised two anti-correlated gene subgroups which contained EP300 and TREX1 respectively and was related to innate immune activation, the C-type lectin receptor signaling pathway and antigen presentation. These two groups correlated strongly with the top 2 principal components from Principal Component Analysis (PCA) conducted on the expression of the 157 unique SNP-heritable immune genes. CD53, CD86 and CYBB, which are highly correlated (p>0.7) to the Thorsson et al. Macrophage Regulation score, were major contributors to PCI while HACD2, LNPEP and EP300, were major contributors to PC2. It was investigated whether this gene correlation would inflate the chance of detecting eQTLs associated with a particular group, however analysis of summary statistics showed that despite their correlation, genes typically did not recover the same SNP associations unless they were encoded at the same genomic locus, such as ERAP1 and LNPEP or OAS1 and OAS3. Finally, to confirm TIME eQTLs were not cancer-type specific, associations with tumor type were conducted. Of the 890 TIME eQTLs, only rs146336885 was associated with tumor type.

Previous studies of germline variation and important modulators of immune checkpoint response, such as APOE, CTSW, CTLA-4, PD-L1, PD-1, CXCR3/CCR5, IRF5 and FGFR4 along with immune signatures and immune cell infiltration have been conducted. These 194 germline associations were incorporated from literature into the analyses (FIG. 2C). Like Shahamatdar et al., immune infiltrates estimated from bulk RNA sequencing were included into the set of immune components that were investigated, however, none of the CIBERSORTx infiltrates passed the SNP-heritability filter. Zhang et al. took a fundamentally different approach, analyzing ER+ breast cancer-associated variants from Michailidou et al. for proximity to immunoinflammatory GWAS variants. The top SNP, rs3903072, was an eQTL for CTSW in breast cancer. Although not specifically focused on breast cancer, the study also identified CTSW as a SNP-heritable IP component (GCTA V(g)/V(p)=12.1%) and detected a pan-cancer association with rs3903072 (beta=0.21, p=2.8e-36). The study by Sayaman et al. focused on 139 immune traits described in the Thorsson et al. paper, of which 106 were immune signatures and 33 included immune measures such as TCR/BCR characteristics, CIBERSORTx infiltration and antigen load. Comparing gene results between Sayaman et al. and the study, 10 genes were shared between the analyses, HLA-DRB5, HLA-B, HLA-DRB1, MICB, HLA-DQB1, HLA-DQB2, HLA-DQA1, HLA-DQA2, MICA, HLA-C, emphasizing the importance of MHC Class I and II machinery in modifying the TIME. Nineteen of the variants were in LD with 361 Sayaman et al. TIME eQTLs (R²>0.50).

Combining the TIME eQTLs and literature associations resulted in a set of 1084 candidate associations. A number of TIME eQTLs were associated with multiple immune genes; thus, there were a greater number of associations than TIME eQTLs. For example, within the discovery pipeline described herein, rs2693076 was associated with LILRB2, PLEK, MYO1F, and CD14. From literature curation, Sayaman et al. identified associations with rs2111485 and multiple signatures, including interferon-signaling and IFIT3 signaling.

### Example 3—Identifying TIME-SNPs Related to Cancer Outcomes

It was next determined if TIME eQTLs could serve as the basis for genetic models for cancer risk, survival and immunotherapy response prediction. An association with gene expression in the TIME does not necessarily mean that the eQTL will impact cancer outcomes. Thus, the TIME eQTLs were evaluated in the context of human cohorts, relying on datasets with both genetic and relevant cancer phenotype data to build models.

For cancer risk, a PheWAS was performed with cancer ICD10 codes in the UK Biobank, and also cross-referenced the associations against summary statistics from the NHGRI-EBI GWAS catalog and Vanderbilt PheWAS catalog. High overlap was observed in risk variants (FDR<0.05) identified by these three sources. When assessing overlap based on the corresponding genes, an even higher degree of overlap was observed, with only 2 eQTLs, TAP2 and LNPEP, being uniquely implicated by the UK Biobank. For survival analysis, TIME eQTLs were evaluated with overall and progression-free survival in the TCGA dataset, treating each tumor type separately. Survival association was evaluated by CoxPH model for tumor types with at least 100 samples available and including covariates relevant to each tumor type.

To investigate the implication of TIME eQTLs for immune checkpoint blockade (ICB) response, sequencing and ICB response information was collected for 276 patients with melanoma treated with immune checkpoint inhibitors from 4 studies, and imputed SNPs from exome sequencing data. Accuracy of exome-based imputation was assessed by comparing original TCGA genotype calls to genotypes imputed in from TCGA exome data at positions matching those in the ICB data; aside from variants on chromosome 6 within the HLA region most were accurately imputed. Ultimately, 525 out of 1084 TIME eQTLs could be imputed with sufficient quality (minor allele frequency>0.05 in all 4 discovery ICB cohorts with imputation accuracy of at least 0.3. Meta-analysis was conducted with METAL using the four melanoma ICB cohorts to evaluate SNP associations with ICB response. No individual eQTLs were significantly associated with ICB response after multiple testing correction.

To model the role of immune genetic background in cancer phenotypes as a whole, polygenic scores were used. The poygenic score construction approach by Elgart et al. was adopted which performs shrinkage based SNP selection followed by construction of a nonlinear, machine learning based PRS capable of capturing interactions between SNPs. For risk analysis, two cancer types were selected for more in depth analysis. The survival analysis was repeated with tumor-type specific polygenic survival score (PSS) as the independent variable. A polygenic ICB score (PICS) was also constructed in the four ICB melanoma cohorts. In each case, genetic models were validated in independent cohorts. These analyses are described herein.

### Example 4—TIME eQTLs Underlying Antigen Presentation Stratify Melanoma and Prostate Cancer Risk

To assess the potential of immune genetic background to influence cancer risk, TIME eQTL derived polygenic risk scores (PRSs) were evaluated in two cancer types with differing levels of immune involvement. Melanoma is classically thought of as an immune ‘hot’ cancer type, with high levels of immune infiltration and one of the highest rates of immunotherapy response. In contrast, prostate cancer tends to have a more suppressed immune microenvironment.

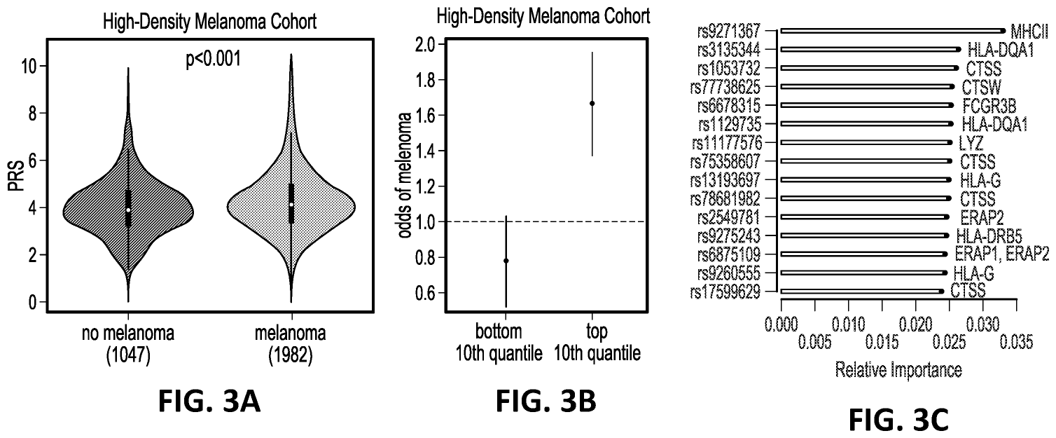

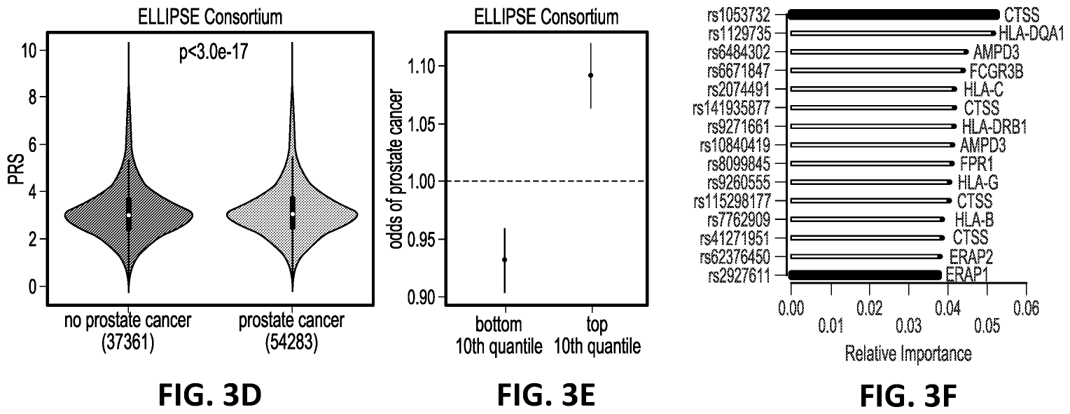

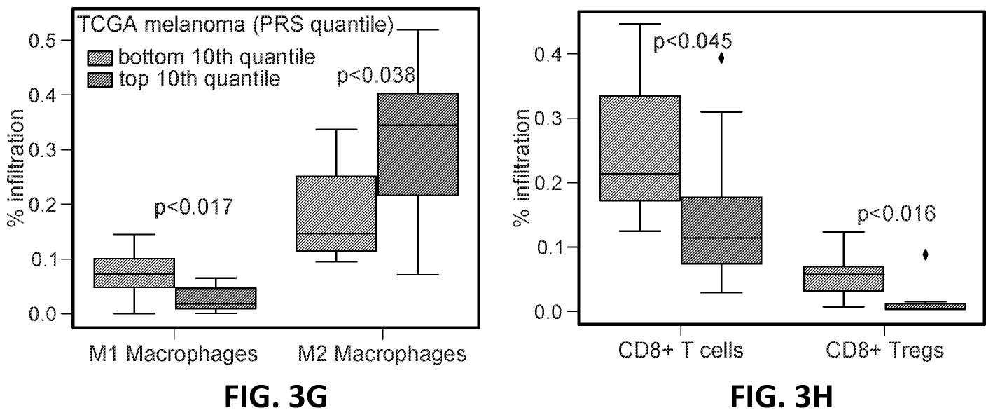

First, PRS from TIME eQTLs in UK Biobank was constructed separately for melanoma and prostate cancer. Because TIME eQTL risk associations were derived in part from the UK Biobank, it was sought to evaluate the resulting PRS models in independent cohorts. The melanoma PRS was evaluated in 3029 melanoma cases and controls from UT MD Anderson (FIG. 3A). As is typical for PRS scores, the difference in score distributions for cases and controls was small (FIG. 3A), but the odds of melanoma were significantly different in the top and bottom 10th quantile in the validation cohort (FIG. 3B). eQTLs related to CTSS and MHC class II genes featured prominently among the most informative features during model fitting, suggesting a role for class II antigen presentation in cancer risk (FIG. 3C). The prostate cancer PRS was validated in a cohort comprising 91,644 cases and controls from the ELLIPSE Consortium with similar results (FIGS. 3D-3E). CTSS and class II MHC genes were once again the most important features, though HLA-B and HLA-C appeared more influential in prostate cancer risk (FIG. 3F). Effect sizes separating the top and bottom quantiles were larger in melanoma than prostate cancer (FIG. 3B vs 3E). While pan-cancer risk analysis implicated individual eQTLs for CTSS, ERAP1, ERAP2, CTSW, and class I and II MHC genes, PRS analysis pointed to additional eQTLs with some shared between melanoma and prostate (FPR1, LYZ, FCGR3B, HLA-G, HLA-H, HLA-DQA1 and HLADQB1), unique to melanoma (MNDA, IL2RA, OAS1, TAP2) or unique to prostate (AMP3D, SIGLEC5, HLA-B, HLA-C, HLA-DRB1).

As the PRS analysis implicated aspects of both antigen directed T cell responses and macrophage activity, it was asked whether the melanoma PRS correlated with T cell and macrophage phenotypes in melanomas in the TCGA dataset. Indeed, tumors in the upper 10th quantile of the melanoma PRS had higher levels of infiltration by pro-tumor inflammatory M2-like (FIG. 3G), but not M0 or M1-like macrophages. Promotion of an inflammatory pro-tumor environment was also correlated with decreased CD8+ T cell infiltration (FIG. 3H). This supports that TIME eQTLs contribute to cancer risk at least in part by modifying the activity of immune cells at the site where a tumor develops.

### Example 5—TIME eQTLs Associated With Survival Implicate Immune Evasion

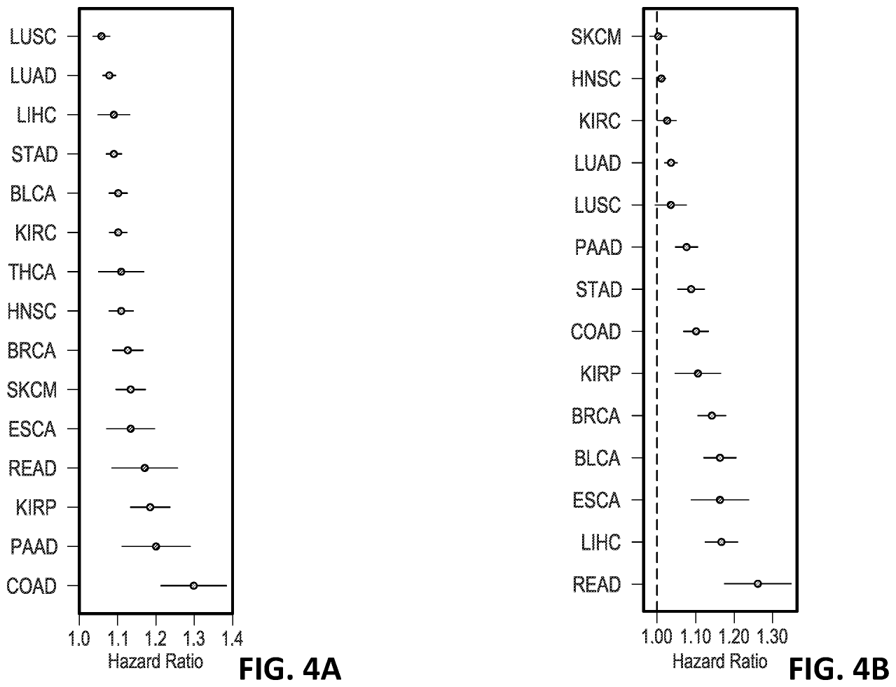

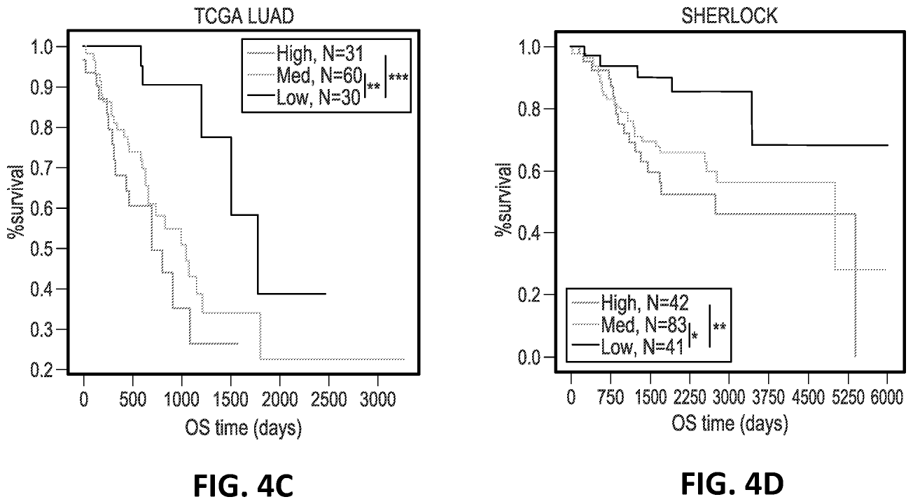

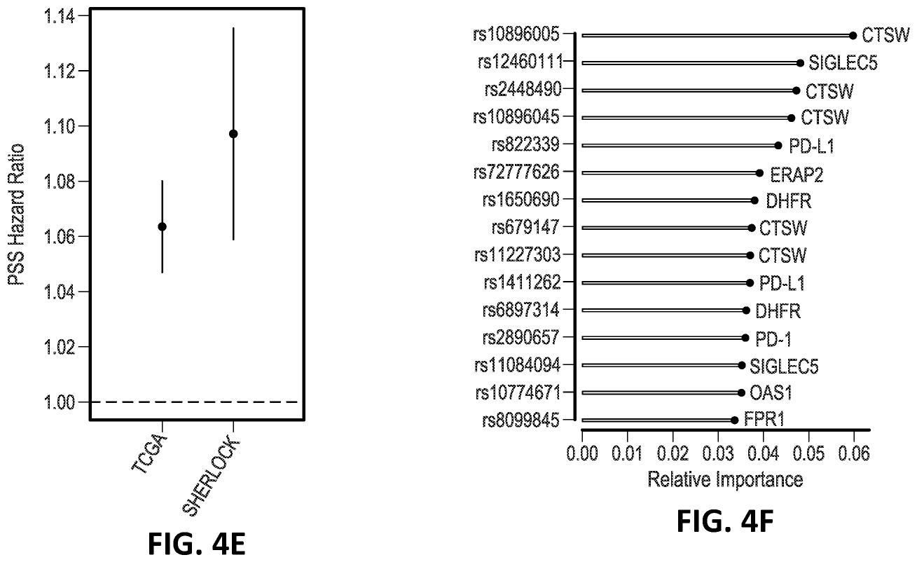

Furthermore, survival associations were revisited to evaluate polygenic contributions. Cancer type-specific PSS was built separately for each tumor type using 70% of samples, then used them to calculate PSS for the remaining 30% of tumors and evaluated these scores along with other covariates in a Cox Proportional-Hazards analysis. Significant associations were found with overall survival in lung adenocarcinoma, breast invasive carcinoma, bladder urothelial carcinoma, clear cell renal carcinoma, papillary renal carcinoma, head and neck squamous cell carcinoma, skin cutaneous melanoma, colorectal adenoma, and stomach adenocarcinoma (FDR<0.05; FIG. 4A) and with progression-free survival in lung adenocarcinoma, breast invasive carcinoma, bladder urothelial carcinoma, rectum adenocarcinoma, colorectal adenocarcinoma, pancreatic adenocarcinoma, stomach adenocarcinoma and hepatocellular carcinoma (FDR<0.05; FIG. 4B).

Among these tumor types, matched survival and genotype data were obtained for 166 non-smokers that developed lung adenocarcinoma from the Sherlock cohort. PRS-stratification of the 30% of TCGA lung adenocarcinoma (LUAD) samples (FIG. 4C) and individuals in the Sherlock cohort (FIG. 4D) showed similar effects on outcome, such that tumors with the lowest PRS scores had the best overall survival. Incorporating the TCGA LUAD-based PRS into a CoxPH analysis of the Sherlock tumors including clinical covariates returned a larger hazard ratio than in the held out 30% of TCGA samples (FIG. 4E). The PRS for overall survival included eQTLs for genes involved in regulating T cell activity (CTSW, PD-1, PD-L1), antigen processing and presentation (VAMP3, ERAP2, MICA), response to immunogenic stimuli such as aberrant DNA or microorganisms (TREX1, OAS1, C3AR1, FPR1), suppression of myeloid cells (SIGLEC5), folate metabolism (GGH, DHFR), amino acid metabolism (CCBL2) and interferon signatures (FIG. 4F). The presence of GGH and DHFR suggested the possibility that our eQTL set could include pharmacogenomic modifiers of anti-folate treatments such as methotrexate and pemetrexed. The validation analysis was revisited, omitting eQTLs for these genes, and found that the PRS still validated in Sherlock.

### Example 6—TIME eQTLs Implicate Targets for Modulating Immune Responses

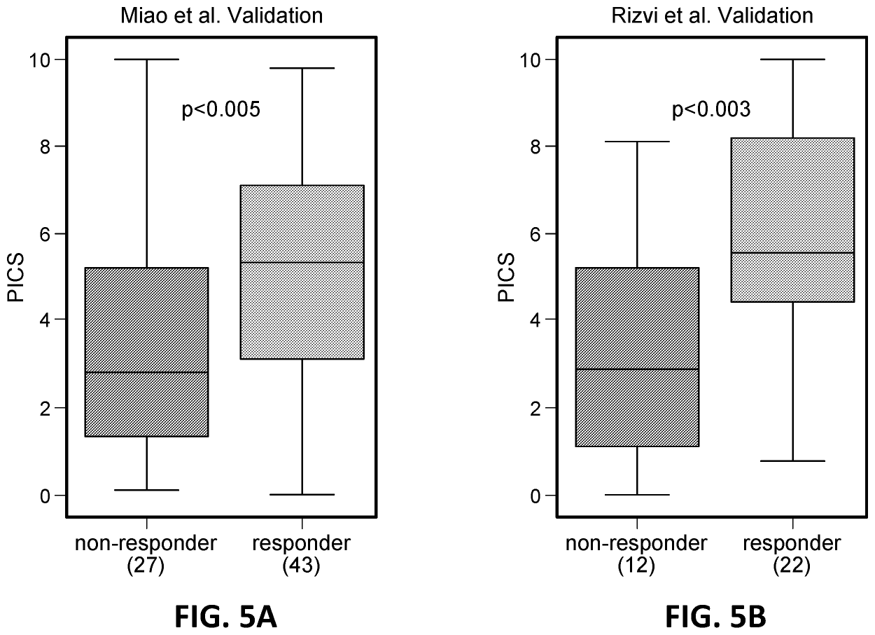

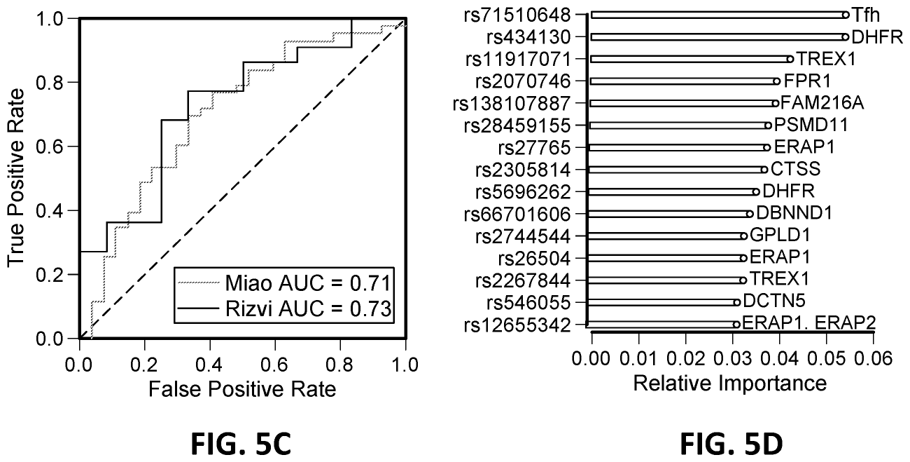

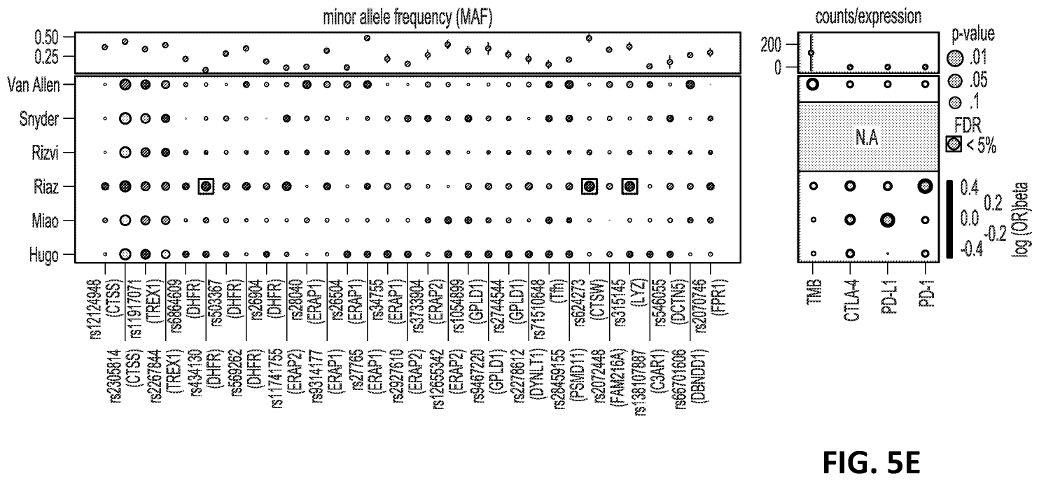

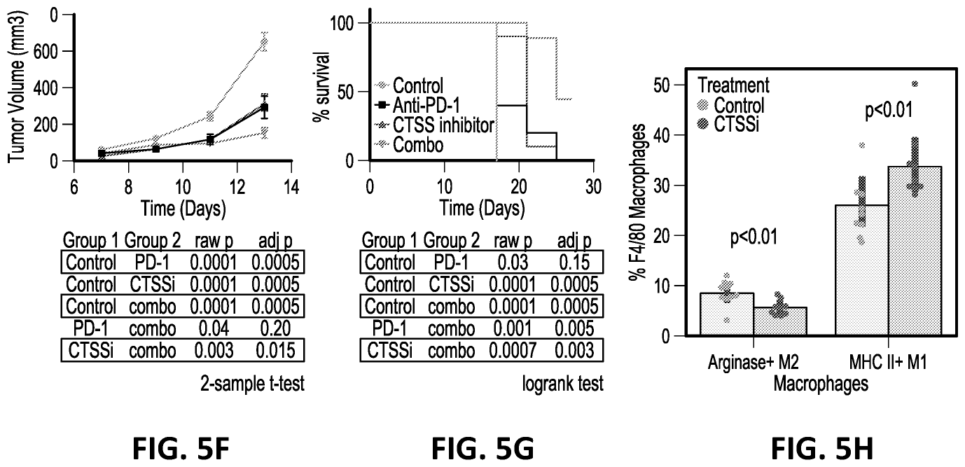

Next, an immunotherapy response-specific PRS was constructed using four published melanoma cohorts treated with immune checkpoint blockade. The predictive potential was validated for this polygenic score in two independent cohorts, one consisting of renal cell carcinomas, and the other of non-small cell lung cancers. In both cohorts, responders had significantly higher polygenic ICB scores (PICS) (FIGS. 5A-5B) and in ROC analysis the PICS achieved an area under the curve greater than 0.7 (FIG. 5C). Feature importance analysis of the PICS model suggested eQTLs involving genes related to DNA replication (TREX1, DHFR) and antigen presentation (PSMD11, ERAP1, ERAP2, CTSS) were most informative (FIG. 5D).

Although tumor-immune interactions vary across tissue sites and tumor characteristics, the study design emphasized tumor-general effects, which may explain the generalization of the PICS across ICB cohorts with distinct tumor types. The PICS selected 30 TIME eQTLs (FIG. 5E), and one SNP associated with Tm infiltration levels. The PICS implicated genes associated with antigen processing and presentation (CTSS, ERAP1, ERAP2, PSMD11), complement (C3AR1) and cytolytic activity (CTSW), vesicular transport (DCTN5, DYNLT1), post-translational regulation (DBNND1, GPLD1), folate metabolism (DHFR), phagocytic activity (FPR1, LYZ) and single stranded DNA response (TREX1). This analysis was repeated selecting 31 TIME eQTLs at random, matched for minor allele frequency, and it was found that the observed difference in burden score between responders and nonresponders was significantly larger than random in both discovery and validation sets.

For most ICB response genes, the direction of effect of variants associated with responder status was mostly consistent across cohorts, though some variants, such as rs28459155 associated with PSMD11 showed less agreement (FIG. 5E). rs28459155 associated with lower odds of being a responder in Miao et al., Rizvi et al. and Hugo et al. but higher odds of being a responder in Van Allen et al., Snyder et al. and Riaz et al. As a comparison to current ICB biomarkers, association of tumor mutation burden (TMB) and expression levels of PD-L1, PD-1, and CTLA-4 was also evaluated with responder status and no significant associations was found (FIG. 5E). Associations were ran with the 31 variants and TMB, PD-L1, PD-1, and CTLA-4 to determine if any variants were associated with these previously researched biomarkers. Only an association between TMB and ERAP1 variant rs27765 and PD-L1 and DHFR variant rs503367 was observed.

PICS-implicated genes were then evaluated as possible entry points to modify anti-tumor immunity. Colocalization of gene expression and GWAS signals can point to putative causal disease-related genes that in the setting of ICB response might suggest candidate targets to stimulate more effective anti-tumor immunity. Examining gene expression data available for 4 out of 6 cohorts, it was noted that none of the 15 genes were significantly differentially expressed between ICB responders and nonresponders. However, some TIME eQTLs were associated with both higher expression of the associated gene and worse ICB response, suggesting that these genes could potentially be inhibited to improve anti-tumor immunity. Of the genes meeting these criteria, only CTSS, TREX1 and PSMD11 had small molecule inhibitors available. For all three genes, the effect of the minor allele on gene expression varied across human cohorts. In the Van Allen cohort where rs11917071 associated with lower odds of being a responder, individuals carrying the minor allele also tended to have increased TREX1 expression. In the Hugo et al. and Miao et al. cohorts, individuals carrying rs2267844 trended toward lower TREX1 expression and higher odds of being a responder. This is consistent with TREX1's role as an immune inhibitor that prevents cGAS-STRING initiation, with inhibition of TREX1 stimulating IFNγ signaling and autoimmunity, making it a potential immunomodulatory target. Individuals with rs28459155 had lower odds of being a responder in 3 of the 6 cohorts and trended toward increased expression of PSMD11 in 2 of these cohorts. A proteosomal protein involved in ubiquitination, PSMD11 is associated with worse prognosis in pancreatic cancer. Individuals with CTSS variant rs23058814 also had higher odds of being a responder and trended toward decreased CTSS expression. Increased CTSS expression has been linked to tumor progression in follicular lymphoma due to decreased CD8+ T cell recruitment. CTSS featured prominently in the cancer risk analysis and, unlike TREX1, had not been implicated as a likely target for solid tumor immunotherapy. Furthermore, increased M1 macrophage infiltration was observed in individuals with the CTSS variant in Hugo et al. suggesting that CTSS activity might contribute to remodeling of the TIME. These considerations led to choosing CTSS as the top target to validate in vivo. Examining two separate mouse immunotherapy-treated mouse models, significant differences in Ctss expression were observed.

To test the hypothesis that inhibition of CTSS would increase anti-tumor immune activity, we treated mice implanted with MC38 tumors with a CTSS small molecule inhibitor. Mice treated with CTSS inhibitors had slowed tumor growth and better survival compared to control mice (FIGS. 5G-5H). The interaction of CTSS inhibitor treatment with anti-PD-1 was also evaluated. Mice treated with CTSS inhibitor or anti-PD-1 monotherapy had significantly decreased tumor growth and better survival compared to control mice. Additionally, tumor growth was further decreased in mice treated with the combination of anti-PD-1 and CTSS inhibitor as compared to mice treated with anti-PD-1 or CTSS inhibitor alone. In the MC38 model, an increase in infiltrating M1 macrophages and a decrease in M2 macrophages was observed similar to findings from Hugo et al. (FIG. 5I). These findings demonstrate that a focused screen for cancer relevant TIME-associated variants provides a fruitful strategy to reveal novel immunotherapy targets. Furthermore, the influence of CTSS inhibition on the myeloid landscape identifies macrophages as potential cell types that may modulate immunotherapy response.

### Example 7—Biological Implications of TIME eQTLs

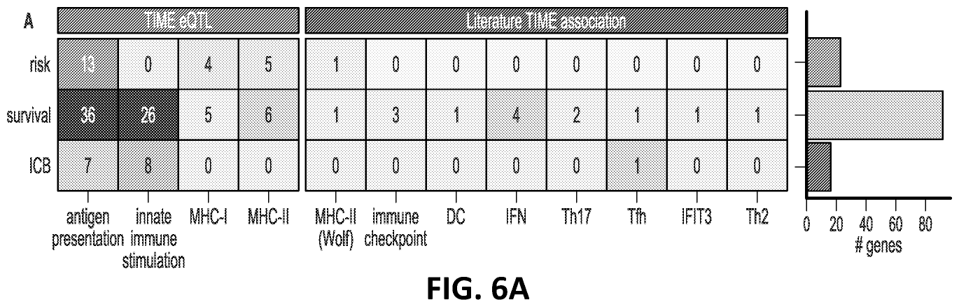

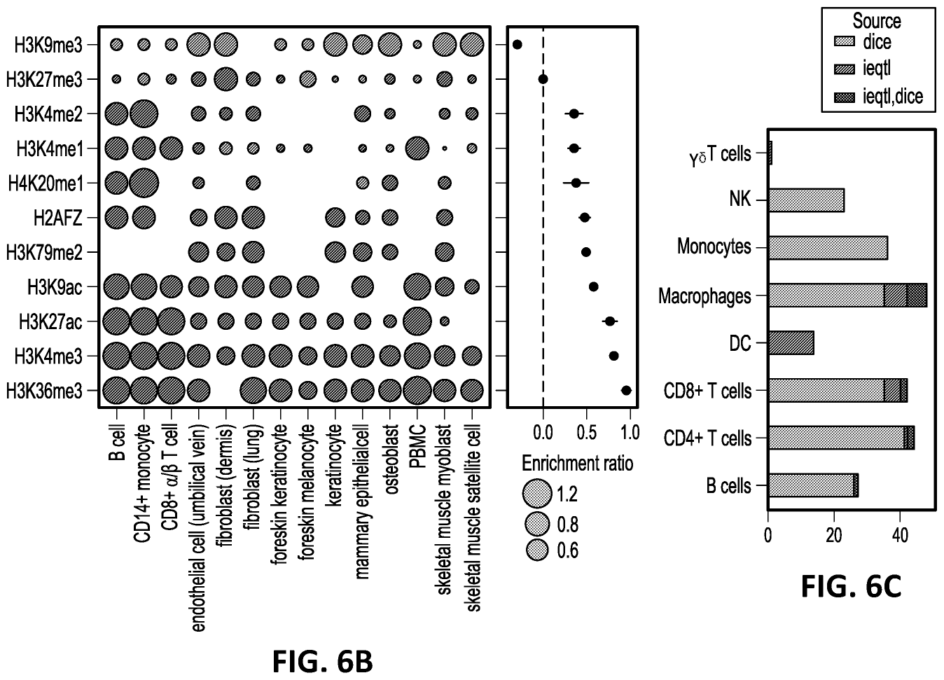

Overall polygenic analysis of cancer-relevant TIME eQTLs implicated 91 genes (counting literature-based signatures as a single gene) as potentially contributing to cancer risk, progression or immunotherapy response (FIG. 6A). From these, it was sought to understand what aspects of the tumor-immune interface were affected. eQTL implicated genes relative to the two broad functional categories established based on gene ontology enrichment analysis of correlated gene groups in the TIME were evaluated. While multiple iQTLs in both categories contributed to survival and ICB associations, genes related to innate immune stimulation (Top GO terms: exogenous peptide antigen processing and presentation, NIK/NF-kβ signaling and C-lectin driven innate immune responses) were notably absent from the risk category. This could reflect differences in the tumor types considered in the risk versus survival analyses performed, or it could reflect that such immune eQTLs only become relevant in later stages of disease, perhaps when the right stimuli are present. Literature associations were also mostly tied to progression, possibly reflecting that many of these were originally reported based on observed effects on prognosis.

The majority of TIME-eQTLs were detected as cis associations (87.1%), aside from 39 (12.9%) trans associations. Eight cancer relevant TIME eQTLs (1.6%) affected protein-coding regions. In the case of HLA-A, HLA-C, FPR1, CTSS<TAP2, missense variants in coding regions were associated with expression differences. In addition, missense variants in PALB2, NOTCH4 and GBP3 were associated with expression differences in DCTN5, MHC Class II and CCBL2, respectively.

As the majority of TIME eQTLs fell within non-coding genomic regions, their potential to affect regulation of chromatin architecture and transcription was evaluated based on histone marks. Regions harboring TIME eQTLs were strongly enriched in H3K27ac, H3K36me3 and H3K4me3 and depleted in H3K9me3 (FIG. 6B). H3K27ac is a known marker of active enhancers and H3K4me3 is usually enriched at promoters near transcription start sites suggesting some TIME eQTLs could affect expression of multiple genes while others may be gene specific. TIME eQTLs were depleted in repressive H3K9me3 marks. Enrichment in histone marks was most pronounced in certain immune cell types.

eQTLs are often cell-type specific, so it was evaluated whether TIME eQTLs in TCGA were dependent on immune cell infiltration level or corresponded to known immune cell-type specific eQTLs in DICE. Macrophages, CD4+ and CD8+ T cells were the most represented cell types. Of the TIME eQTLs, 48 influenced gene expression in macrophage, 44 were CD4+ T cell eQTLs, 42 were CD8+ T cell eQTLs and 27 were B cell eQTLs (FIG. 6C). Comparing myeloid-specific eQTLs to lymphoid-specific eQTLs, variants associated with FAM216A, RNASE6, MARCH1, OAS1, HLA-DQB2, GPNMB, LYZ and CPVL were myeloid-specific.

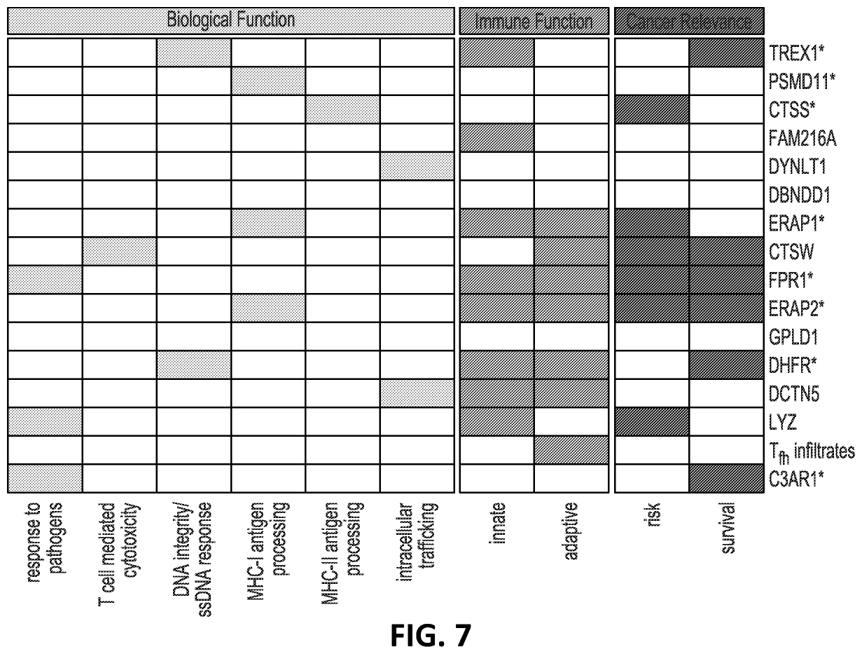

Re-visiting the 15 genes implicated by the PICS model (FIG. 7), it was sought to gain more perspective on the aspects of immunity influential for immunotherapy response. Many of these genes also had risk or survival associated eQTLs and were modifiers of gene expression in various immune cell types. Peptide processing appeared to be a major factor contributing to ICB responses, Peptidases involved in both class I (ERAP1, ERAP2) and class II (CTSS) peptide processing appeared to be a shared component between ICB response and risk. In contrast, aspects relating to cytolytic activity (CTSW), pathogen responses (FPR1, C3A1 and LYZ) and single stranded DNA responses (TREX1) shared more in common between ICB response and progression. In contrast, eQTLs involving intracellular trafficking proteins DCTN5 and DYNLT1 appeared to uniquely affect ICB response. Interestingly, eQTLs for DCTN5 showed immune cell type specific effects, whereas those for DYNLT1 did not. These proteins mediate vesicle and organelle trafficking that may have different implications in different cell types. For example, in T cells they may play a role in immune synapse formation and energetics by transporting mitochondria to the membrane. Interestingly, another vesicle trafficking gene, VAMP3 was implicated in progression. Altogether, the analyses reveal a subset of TIME eQTLs that highlight key aspects of immune function with implications for cancer risk, progression and immunotherapy response.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Exemplary Embodiments

Embodiment 1. A method of identifying an immunotherapy target, the method comprising:
(a) select a plurality of tumor immune microenvironment (TIME) genes;
(b) identify a cancer relevant TIME gene from the plurality of TIME genes; and
(c) determine response of the cancer relevant TIME gene to immunotherapy, thereby identifying an immunotherapy target.
   Embodiment 2. The method of embodiment 1, wherein a TIME gene comprises an immunomodulator, an antigen presentation gene, an immune checkpoint gene, or an immune cell-type marker gene.
   Embodiment 3. The method of embodiment 1 or 2, wherein the TIME gene is located in the HLA region.
   Embodiment 4. The method of embodiment 1 or 2, wherein the TIME gene is located outside the HLA region.
   Embodiment 5. The method of any one of embodiments 1-4, wherein the cancer relevant TIME gene comprises a germline variant.
   Embodiment 6. The method of embodiment 5, wherein the germline variant comprises a single nucleotide polymorphism (SNP).
   Embodiment 7. The method of any one of embodiments 1-6, wherein the cancer relevant TIME gene comprises LAIR1, TREX1, CTSS, CTSW, or LILRB2.
   Embodiment 8. The method of any one of embodiments 1-7, wherein the cancer relevant TIME gene comprises CTSS.
   Embodiment 9. The method of any one of embodiments 1-8, wherein the cancer relevant TIME gene is relevant to a solid tumor cancer.
   Embodiment 10. The method of embodiment 9, wherein the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.
   Embodiment 11. The method of embodiment 10, wherein the solid tumor cancer is follicular lymphoma.
   Embodiment 12. A method of predicting a subject's response to immunotherapy, the method comprising:
(a) select a plurality of tumor immune microenvironment (TIME) genes;
(b) identify a cancer relevant TIME gene from the plurality of TIME genes;
(c) determine response of the cancer relevant TIME gene to immunotherapy; and
(d) detecting the cancer relevant TIME gene in a biological sample from the subject, thereby predicting the subject's response to immunotherapy.
   Embodiment 13. The method of embodiment 12, wherein a TIME gene comprises an immunomodulator, an antigen presentation gene, an immune checkpoint gene, or an immune cell-type marker gene.
   Embodiment 14. The method of embodiment 12 or 13, wherein the TIME gene is located in the HLA region.
   Embodiment 15. The method of embodiment 12 or 13, wherein the TIME gene is located outside the HLA region.
   Embodiment 16. The method of any one of embodiments 12-15, wherein the cancer relevant TIME gene comprises a germline variant.
   Embodiment 17. The method of embodiment 16, wherein the germline variant comprises a single nucleotide polymorphism (SNP).
   Embodiment 18. The method of any one of embodiments 12-17, wherein the cancer relevant TIME gene comprises LAIR1, TREX1, CTSS, CTSW, or LILRB2.
   Embodiment 19. The method of any one of embodiments 12-18, wherein the cancer relevant TIME gene comprises CTSS.
   Embodiment 20. The method of any one of embodiments 12-19, wherein the cancer relevant TIME gene is relevant to a solid tumor cancer.
   Embodiment 21. The method of embodiment 20, wherein the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.
   Embodiment 22. The method of embodiment 21, wherein the solid tumor cancer is follicular lymphoma.
   Embodiment 23. The method of any one of embodiments 12-22, wherein the biological sample comprises a tissue, a tissue section, an organ, an organism, an organoid, or a cell culture sample
   Embodiment 24. A method of predicting a subject's response to immunotherapy, the method comprising:
(a) obtaining a biological sample from the subject; and
(b) detecting a cancer relevant tumor immune microenvironment (TIME) gene in the biological sample, thereby predicting the subject's response to immunotherapy.
   Embodiment 25. The method of embodiment 24, wherein the cancer relevant TIME gene comprises a germline variant.
   Embodiment 26. The method of embodiment 25, wherein the germline variant comprises a single nucleotide polymorphism (SNP).
   Embodiment 27. The method of any one of embodiments 24-26, wherein the cancer relevant TIME gene comprises LAIR1, TREX1, CTSS, CTSW, or LILRB2.
   Embodiment 28. The method of any one of embodiments 24-27, wherein the cancer relevant TIME gene comprises CTSS.
   Embodiment 29. The method of any one of embodiments 24-28, wherein the cancer relevant TIME gene is relevant to a solid tumor cancer.
   Embodiment 30. The method of embodiment 29, wherein the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.
   Embodiment 31. The method of embodiment 30, wherein the solid tumor cancer is follicular lymphoma.
   Embodiment 32. The method of any one of embodiments 24-31, wherein the biological sample comprises a tissue, a tissue section, an organ, an organism, an organoid, or a cell culture sample.
   Embodiment 33. A method of predicting a subject's response to immunotherapy, the method comprising:
(a) obtaining a biological sample from the subject, wherein the subject has a disease; and
(b) detecting a cancer relevant tumor microenvironment (TIME) gene in the biological sample, wherein the cancer relevant TIME gene is CTSS, thereby predicting the subject's response to immunotherapy.
   Embodiment 34. The method of embodiment 33, wherein the disease is a cancer.
   Embodiment 35. The method of embodiment 34, wherein the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.
   Embodiment 36. The method of any one of embodiments 33-35, wherein the biological sample comprises a tissue, a tissue section, an organ, an organism, an organoid, or a cell culture sample.
   Embodiment 37. A method of treating a solid tumor in a subject, the method comprising:
(a) obtaining a biological sample from the subject, and
(b) detecting a cancer relevant tumor microenvironment (TIME) gene in the biological sample, wherein the detection of the cancer relevant TIME gene is used to predict the subject's response to a treatment; and
(c) administering the treatment, thereby treating the solid tumor in the subject.
   Embodiment 38. The method of embodiment 37, wherein the cancer relevant TIME gene comprises a germline variant.
   Embodiment 39. The method of embodiment 38, wherein the germline variant comprises a single nucleotide polymorphism (SNP).
   Embodiment 40. The method of any one of embodiments 37-39 wherein the cancer relevant TIME gene comprises LAIR1, TREX1, CTSS, CTSW, or LILRB2.
   Embodiment 41. The method of any one of embodiments 37-40, wherein the cancer relevant TIME gene comprises CTSS.
   Embodiment 42. The method of any one of embodiments 37-41, wherein the cancer relevant TIME gene is relevant to a solid tumor cancer.
   Embodiment 43. The method of embodiment 42, wherein the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.
   Embodiment 44. The method of embodiment 43, wherein the solid tumor cancer is follicular lymphoma.
   Embodiment 45. The method of any one of embodiments 37-44, wherein the treatment is immunotherapy.
   Embodiment 46. The method of any one of embodiments 37-45, wherein the biological sample comprises a tissue, a tissue section, an organ, an organism, an organoid, or a cell culture sample.

1. A method of improving sensitivity to immune checkpoint blockade (ICB) in a subject with a solid tumor cancer, the method comprising:
administering to the subject with a solid tumor cancer an inhibitor of a tumor immune microenvironment (TIME) gene, wherein the TIME gene is selected from the group consisting of SLC11A1, CCBL2, LILRB2, LILRB4, LAIR1, CTSS, CTSW, B2M, HAUS1, MS4A4A, FCGR2B, LYZ, and GNPTAB,
thereby improving sensitivity to ICB in the subject with a solid tumor cancer.

2. The method of claim 1, wherein the TIME gene comprises a single nucleotide polymorphism (SNP).

3. The method of claim 1, wherein the TIME gene comprises CTSS.

4. The method of claim 1, wherein the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma or prostate cancer.

5. The method of claim 4, wherein the solid tumor cancer is follicular lymphoma.

6. A method of treating a solid tumor cancer in a subject by detecting a germline tumor immune microenvironment-single nucleotide polymorphism (TIME-SNP) of a TIME gene in a subject, the method comprising:
(a) administering to the subject an inhibitor of the TIME gene selected from the group consisting of SLC11A1, CCBL2, LILRB2, LILRB4, LAIR1, CTSS, CTSW, B2M, HAUS1, MS4A4A, FCGR2B, LYZ, and GNPTAB; and
(b) administering to the subject an immune checkpoint blockade (ICB) treatment, thereby treating the tumor in the subject.

7. The method of claim 6, wherein the TIME gene comprises CTSS.

8. The method of claim 6, wherein the TIME gene is relevant to the solid tumor cancer.

9. The method of claim 6, wherein the solid tumor cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma or prostate cancer.

10. The method of claim 9, wherein the solid tumor cancer is follicular lymphoma.

11. The method of claim 6, wherein the inhibitor of the TIME gene and the ICB treatment are administered simultaneously.

12. The method of claim 6, wherein the inhibitor of the TIME gene is administered prior to the ICB treatment.
